(19) United States
(12) Patent Application Publication
(10) Pub. No.: US 2023/0233803 A1
(43) Pub. Date: Jul. 27, 2023

(54) Systems and Methods for Adjusting the Length of Line Draw Lever Lock Components for Compatibility With Various Length Peripheral Intravenous Catheters (PIVC)

(71) Applicant: Becton, Dickinson and Company, Franklin Lakes, NJ (US)

(72) Inventors: John M. Lackey, West Valley City, UT (US); Megan S. Scherich, Salt Lake City, UT (US); Christopher Quach, North Salt Lake, UT (US); Taylor Matthew Eley, Alpharetta, GA (US); Justin G. Hortin, Farmington, UT (US)

(21) Appl. No.: 18/099,541

(22) Filed: Jan. 20, 2023

Related U.S. Application Data
(60) Provisional application No. 63/301,728, filed on Jan. 21, 2022.

(51) Int. Cl. A61M 25/01; A61M 25/00

(52) U.S. Cl. CPC A61M 25/0113; A61M 25/0097; A61M 25/0606

(57) ABSTRACT

A fluid transfer device for use with a peripheral intravenous catheter (PIVC) including an introducer body having a proximal end and a distal end, and a flow tube at least partially housed within the introducer body, wherein the flow tube is configured to be selectively extendable from the introducer body. The fluid transfer device also includes a lever lock coupled to the distal end of the introducer body, wherein the lever lock includes a lumen configured to allow the flow tube to pass therethrough, and wherein an effective length of the lever lock relative to the distal end of the introducer body is adjustable in order to accommodate use of the fluid transfer device with indwelling catheters of the PIVC having various lengths.

### CROSS-REFERENCE TO RELATED APPLICATION

The present application claims priority to U.S. Provisional Application Ser. No. 63/301,728, entitled “Systems and Methods for Adjusting the Length of Line Draw Lever Lock Components for Compatibility with Various Length Peripheral Intravenous Catheters (PIVC)”, filed Jan. 21, 2022, the entire disclosure of which is hereby incorporated by reference in its entirety.

### BACKGROUND OF THE INVENTION

### Field of the Invention

The present disclosure generally relates to blood draw instruments and related assemblies, systems, and methods for use with peripheral intravenous catheters (PIVCs). The blood draw instruments are configured to be adjustable in effective length in order to allow for blood collection via PIVCs having indwelling catheters of various lengths.

### Description of Related Art

A catheter is commonly used to infuse fluids into vasculature of a patient. For example, the catheter may be used for infusing normal saline solution, various medicaments, or total parenteral nutrition. Furthermore, the catheter may also be used for withdrawing blood from the patient.

The catheter may be an over-the-needle peripheral intravenous catheter (PIVC). In this case, the catheter may be mounted over an introducer needle having a sharp distal tip. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient. After proper placement of the needle, the clinician may temporarily occlude flow in the vasculature and remove the needle, leaving the catheter in place (i.e., “indwelled”) for future blood withdrawal and/or fluid infusion.

In order to complete blood draws from PIVCs having indwelled catheters, fluid transfer devices have been developed, with the fluid transfer devices configured to overcome previous challenges related to blood draw through PIVCs such as, e.g., the possibility of catheter collapse, reduced blood flow due to debris built up on or within the catheter, etc. One such device, PIVO™ from Velano Vascular, Inc., is configured as a single-use device which temporarily attaches to a PIVC to draw a blood sample. Using an existing peripheral intravenous line as a conduit to the vasculature, the PIVO™ device advances a flexible, internal flow tube through the PIVC, beyond the catheter tip, and into the vein to collect a blood sample. This flow tube is designed to extend beyond the suboptimal draw conditions around the indwelling line to reach vein locations where blood flow is optimal for aspiration. Once blood collection is complete, the flow tube is retracted, and the device is removed from the PIVC and discarded. An example of one such fluid transfer device is shown and described in U.S. Pat. No. 10,300,247 B2, which is incorporated by reference herein in its entirety.

Referring to FIG. 1, an example of a PIVC system 100 utilizing a fluid transfer device as known in the art is shown. PIVC system 100 includes a catheter adapter 102 from which an indwelling catheter 118 distally extends a distance A. The catheter adapter 102 may include a Y-adapter, a straight adapter, or another suitable adapter. The catheter adapter 102 further includes a hub 114, and the catheter system 100 may include a fluid transfer device 104 having a lever lock 108, an introducer 110, and an actuator 112. The lever lock 108 may include a pair of deflectable arms 116A, 116B which allow for selective attachment and detachment of the lever lock 108 from the hub 114 of catheter adapter 102. Furthermore, the lever lock 108 may include a coupler 117 for coupling the introducer 110 to the lever lock 108.

As is described in, e.g., U.S. Pat. No. 10,300,247 B2, the introducer 110 is configured to house a flexible flow tube 120, which may be linearly fed through the indwelling catheter 118 via the actuator 112 such that at its full extension, a distal end of flow tube 120 extends beyond a distal end of indwelling catheter 118 by a distance B. In this way, the flow tube 120 is able to access the patient's vasculature beyond the indwelling catheter 118, providing for improved blood draw through the catheter adapter 102. The flow tube 120 may be fluidly coupled to, e.g., a tube holder 106 via an extension tube 107 having a clamp 109 so as to enable blood draw via the PIVC system 100 into, e.g., an evacuated tube inserted into the holder 106. After blood draw is complete, the flow tube 120 can be retracted, and the fluid transfer device 104 can be decoupled from the catheter adapter 102.

However, in some instances, PIVCs are offered with indwelling catheters of varying lengths, with the specific length being selected based on, e.g., patient anatomy, application, etc. For example, the BD NEXIVA™ Closed IV Catheter System from Becton Dickinson, and Company provides 20 gauge PIVCs with catheter lengths of 1.00 in., 1.25 in., and 1.75 in., respectively. While these catheters having various lengths may allow for improved placement of the PIVC into a patient's vasculature, they may prove problematic when utilized with a fluid transfer device configured for blood collection via the PIVC. Specifically, as described above with respect to FIG. 1, the flow tube of the fluid transfer device typically has a maximum extension length to which it can be advanced beyond the indwelling catheter's distal tip. While that maximum length of the flow tube may be sufficient for suitable protrusion from a PIVC having a catheter length of, e.g., 1.00 in., it may not provide adequate protrusion from a PIVC having a catheter length of, e.g., 1.75 in. Conversely, if the fluid transfer device were designed to provide suitable protrusion from a PIVC having a longer catheter length (e.g., 1.75 in.), the flow tube of that same device may extend an undesirable distance beyond the tip of a shorter (e.g., 1.00 in.) catheter.

### SUMMARY OF THE INVENTION

Accordingly, the present disclosure generally relates to a fluid transfer devices used for blood draw via PIVCs and related assemblies, systems, and methods. The effective length of the fluid transfer devices may be selectable and/or adjustable such that the length of the flow tube extending from the fluid transfer device is compatible with indwelling catheters of varying length.

In accordance with an embodiment of the present application, a fluid transfer device for use with a peripheral intravenous catheter (PIVC) includes an introducer body having a proximal end and a distal end, and a flow tube at least partially housed within the introducer body, wherein the flow tube is configured to be selectively extendable from the introducer body. The fluid transfer device also includes a lever lock coupled to the distal end of the introducer body, wherein the lever lock includes a lumen configured to allow the flow tube to pass therethrough, and wherein an effective length of the lever lock relative to the distal end of the introducer body is adjustable in order to accommodate use of the fluid transfer device with indwelling catheters of the PIVC having various lengths.

In certain configurations, the lever lock includes an elongated coupler portion, a pair of deflectable arms, and a blunt cannula. The pair of deflectable arms may be configured to releasably couple the lever lock to a hub of a catheter adapter of the PIVC. At least a portion of the elongated coupler portion may be configured to be slidable within at least a distal portion of the introducer body. The distal portion of the introducer body may taper so as to restrict an amount in which the elongated coupler portion of the lever lock may slide relative to the distal portion of the introducer.

In certain configurations, the elongated coupler portion of the lever lock may include a plurality of external threads, and a distal portion of the introducer body may include a plurality of internal threads engageable with the external threads of the elongated coupler portion. The fluid transfer device may also include a spring-biased pin coupled to the elongated coupler portion of the lever lock. The introducer body may also include at least two openings formed therein, and the spring-biased pin of the elongated coupler portion of the lever lock may be configured to selectively engage with the at least two openings of the introducer body. A cantilever slider member may be coupled to a proximal portion of the elongated coupler portion of the lever lock, wherein the cantilever slider member includes a pair of engagement portions.

Optionally, the introducer body may further include at least two opening pairs formed therein, and wherein the pair of engagement portions of the cantilever slider member are configured to selectively engage with the at least two opening pairs of the introducer body. The fluid transfer device may also include an actuator member movable relative to the introducer body, wherein the actuator member is configured to feed the flow tube through the introducer body and the lever lock. The proximal end of the introducer body may include a threaded interface.

In accordance with another embodiment of the invention, a fluid transfer device for use with a peripheral intravenous catheter (PIVC)includes an introducer body having a proximal end and a distal end, a flow tube at least partially housed within the introducer body, wherein the flow tube is configured to be selectively extendable from the introducer body, and a first lever lock selectively couplable to the distal end of the introducer body. The first lever lock may include a first lumen configured to allow the flow tube to pass therethrough. The fluid transfer device may also include a second lever lock selectively couplable to the distal end of the introducer body, wherein the second lever lock includes a second lumen configured to allow the flow tube to pass therethrough. A length of the first lever lock may be shorter than a length of the second lever lock in order to accommodate use of the fluid transfer device with indwelling catheters of the PIVC having various lengths.

In certain configurations, each of the first lever lock and the second lever lock includes an elongated coupler portion, a pair of deflectable arms, and a blunt cannula. The pair of deflectable arms may be configured to releasably couple the lever lock to a hub of a catheter adapter of the PIVC.

In certain configurations, the first lever lock is configured for use with indwelling catheters having a length of 1.00 inches, and the second lever lock is configured for use with indwelling catheters having a length of 1.75 inches.

In accordance with another embodiment of the present invention, a fluid transfer device for use with a peripheral intravenous catheter (PIVC) includes an introducer body having a proximal end and a distal end, a flow tube at least partially housed within the introducer body, wherein the flow tube is configured to be selectively extendable from the introducer body, and a lever lock coupled to the distal end of the introducer body. The lever lock may include an elongated coupler portion, and further the elongated coupler portion of the lever lock is telescopically slidable within at least a distal portion of the introducer body in order to accommodate use of the fluid transfer device with indwelling catheters of the PIVC having various lengths.

In certain configurations, the lever lock further includes a pair of deflectable arms and a blunt cannula, and wherein the pair of deflectable arms are configured to releasably couple the lever lock to a hub of a catheter adapter of the PIVC. The fluid transfer device may also include a spring-biased pin coupled to the elongated coupler portion of the lever lock, wherein the introducer body further includes at least two openings formed therein, and wherein the spring-biased pin of the elongated coupler portion of the lever lock is configured to selectively engage with the at least two openings of the introducer body.

In certain configurations, the fluid transfer device further includes a cantilever slider member coupled to a proximal portion of the elongated coupler portion of the lever lock, wherein the cantilever slider member comprises a pair of engagement portions, wherein the introducer body further includes at least two opening pairs formed therein, and wherein the pair of engagement portions of the cantilever slider member are configured to selectively engage with the at least two opening pairs of the introducer body.

Further details and advantages of the invention will become clear upon reading the following detailed description in conjunction with the accompanying drawing figures, wherein like parts are designated with like reference numerals throughout.

### BRIEF DESCRIPTION OF THE DRAWINGS

FIG. 1 illustrates a PIVC system incorporating a fluid transfer device for blood collection in accordance with the prior art;

FIG. 2A illustrates a partial cross-sectional view of a fluid transfer device for blood collection having a lever lock in a first configuration in accordance with an aspect of the present disclosure;

FIG. 2B illustrates a partial cross-sectional view of the fluid transfer device of FIG. 2A having a lever lock in a second configuration;

FIG. 3A illustrates a partial cross-sectional view of a fluid transfer device for blood collection having a lever lock in a first configuration in accordance with another aspect of the present disclosure;

FIG. 3B illustrates a partial cross-sectional view of the fluid transfer device of FIG. 3A having a lever lock in a second configuration;

FIG. 4A illustrates a partial cross-sectional view of a fluid transfer device for blood collection having a lever lock in a first configuration in accordance with another aspect of the present disclosure;

FIG. 4B illustrates a partial cross-sectional view of the fluid transfer device of FIG. 4A having a lever lock in a second configuration;

FIG. 5 illustrates a partial cross sectional view of a fluid transfer device for blood collection having a lever lock in first configuration in accordance with another aspect of the present disclosure;

FIG. 6A illustrates a partial cross-sectional view of a fluid transfer device for blood collection having a lever lock in a first configuration in accordance with another aspect of the present disclosure;

FIG. 6B illustrates a partial cross-sectional view of the fluid transfer device of FIG. 6A having a lever lock in a second configuration;

FIG. 7A illustrates a partial cross-sectional view of a fluid transfer device for blood collection having a lever lock of a first size in accordance with another aspect of the present disclosure; and

FIGS. 7B illustrates a partial cross-sectional view of a lever lock of a second size for use with the fluid transfer device of FIG. 7A.

### DESCRIPTION OF PREFERRED EMBODIMENTS

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

Embodiments of the present disclosure will primarily be described in the context of fluid transfer devices for use with PIVCs. However, embodiments of the present disclosure equally extend to use with other catheter devices.

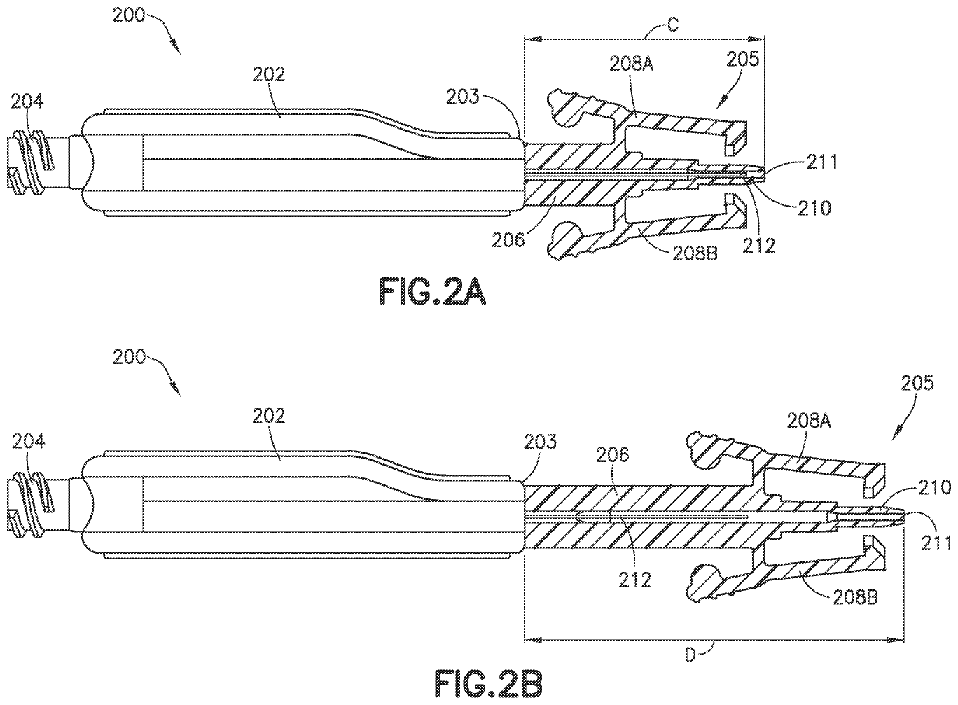

Referring to FIGS. 2A and 2B, a fluid transfer device 200 for use in conjunction with, e.g., a PIVC is shown. Fluid transfer device 200 includes an introducer body 202 having a distal end 203 and a proximal end 204. The proximal end 204 may be configured to include an interface (e.g., a threaded interface) for coupling the fluid transfer device 200 to, e.g., a luer lock access device (LLAD) for performing blood collection. However, it is to be understood that the proximal end 204 may have any appropriate interface and/or coupling means.

While not shown, it is to be understood that fluid transfer device 200 may be configured to include an actuator member movable with respect to the introducer body 202. In some embodiments, the actuator may be configured to be slidable along at least part of the length of the introducer body 202, with the actuator also being coupled to a flexible flow tube 212 such that movement of the actuator along the introducer body 202 results in corresponding linear movement of the flow tube 212 relative to the introducer body 202. However, other forms of actuators and/or methods of actuation of the flow tube 212 relative to the introducer body 202 may also be utilized in accordance with the present disclosure.

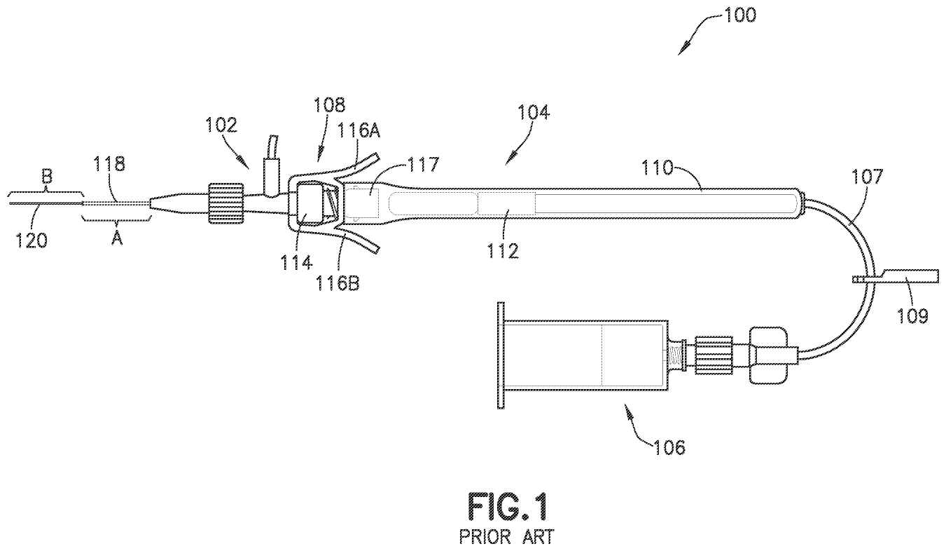

Referring still to FIGS. 2A and 2B, the fluid transfer device 200 also includes a lever lock 205. Lever lock 205 is configured to removably couple the fluid transfer device 200 to, e.g., the hub of a catheter adapter, as is shown and described above with respect to FIG. 1. Deflectable arms 208A, 208B enable such selective connection to the hub of a catheter adapter, and a blunt cannula 210 extends from a distal end of the lever lock 205, with the blunt cannula 210 including a lumen 211 extending therethrough so as to fluidly couple the fluid transfer device 200 and the catheter adapter (not shown) when interconnected.

The lever lock 205 further includes an elongated coupler portion 206 extending from the distal end 203 of introducer body 202. In some embodiments, the coupler portion 206 is substantially cylindrical and surrounds the lumen 211. However, it is to be understood that coupler portion 206 need not be cylindrical, and may be formed of any appropriate shape or shapes. The coupler portion 206 may be joined with the introducer body 202 via any appropriate means. In some embodiments, the coupler portion 206 may be fixedly attached to introducer body 202, while in other embodiments, the coupler portion 206 may be movably coupled to introducer body 202.

As is noted above, the flow tube 212 of fluid transfer device 200 is configured to be extendable (and retractable) from the introducer body 202 and through the lumen 211 so as to be selectively positioned within a patient's vasculature beyond a distal end of an indwelling catheter, thereby enabling blood draw through the fluid transfer device 200. However, due to variations in the length of indwelling catheters, in some instances, a flow tube may not sufficiently extend beyond the distal end of the indwelling catheter or, alternatively, may extend an undesirable distance beyond the distal end of the indwelling catheter. Thus, in accordance with an aspect of the present disclosure, and as shown in FIGS. 2A and 2B, fluid transfer device 200 is configured such that the length of coupler portion 206 of the lever lock 205 may be varied so as to accommodate varying lengths of indwelling catheters. For example, referring to FIG. 2A, the lever lock 205 has a length C extending from the distal end 203 of introducer body 202 to the distal end of blunt cannula 210. However, referring to FIG. 2B, the lever lock 205 has a length D extending from the distal end 203 of introducer body 202 to the distal end of blunt cannula 210, with length D being greater than length C. To provide for such varied overall lengths, the lever lock 205 may be provided with a coupler portion 206 of varied lengths, as is shown in FIGS. 2A and 2B.

Accordingly, fluid transfer device 200 may be configured for usage with indwelling catheters having a variety of lengths. For example, referring to FIG. 2A, lever lock 205 having an external overall length C may be utilized with longer indwelling catheters (e.g., 1.75 inch catheters), as the shorter lever lock length allows for a greater length of flow tube 212 to be fed from the fluid transfer device 200, through the catheter adapter, and through (and beyond) the indwelling catheter. Conversely, referring to FIG. 2B, lever lock 205 having an external overall length D may be utilized with shorter indwelling catheters (e.g., 1.00 inch catheters), as the longer lever lock 205 allows a shorter length of flow tube 212 to be fed from the fluid transfer device 200. In this way, the dimensions of the introducer body 202 and/or flow tube 212 need not change in order to accommodate various lengths of indwelling catheters.

In some embodiments, the fluid transfer device 200 may be provided with the lever lock 205 fixedly coupled to the distal end 203 of introducer body 202. Thus, in such embodiments, the fluid transfer device 200 having a lever lock 205 of a certain length may be specifically selected based upon a known indwelling catheter length. Alternatively, in other embodiments, the lever lock 205 may be selectively decoupled from the introducer body 202, thereby enabling a user to select a lever lock 205 of an appropriate length while using a common introducer body 202.

Furthermore, while FIGS. 2A and 2B illustrate lever lock 205 having two differing lengths, it is to be understood that lever lock 205 may be provided with more than two possible lengths so as to accommodate other indwelling catheter lengths such as, e.g., 1.25 inches.

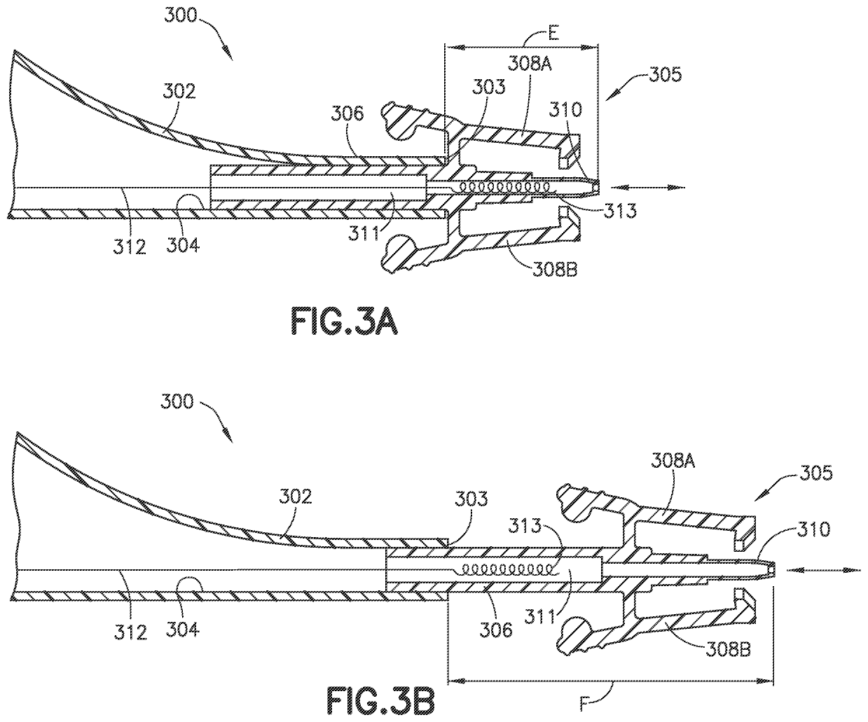

Next, referring to FIGS. 3A and 3B, a fluid transfer device 300 in accordance with another aspect of the present disclosure is shown. Fluid transfer device 300 includes an introducer body 302 having a distal end 303. While not shown, it is to be understood that introducer body 302 further includes a proximal end, and the proximal end may be configured to include an interface (e.g., a threaded interface) for coupling the fluid transfer device 300 to, e.g., a luer lock access device (LLAD) for performing blood collection. However, it is to be understood that the proximal end of introducer body 302 may have any appropriate interface and/or coupling means.

Furthermore, similar to fluid transfer device 200 described above with respect to FIGS. 2A and 2B, it is to be understood that fluid transfer device 300 may be configured to include an actuator member movable with respect to the introducer body 302. In some embodiments, the actuator may be configured to be slidable along at least part of the length of the introducer body 302, with the actuator also being coupled to a flexible flow tube 312 such that movement of the actuator along the introducer body 302 results in corresponding linear movement of the flow tube 312 relative to the introducer body 302. However, the actuator is not limited to such a slidable configuration, and any suitable actuator mechanism may be utilized.

The fluid transfer device 300 also includes a lever lock 305. Lever lock 305 is configured to removably couple the fluid transfer device 300 to, e.g., the hub of a catheter adapter. Deflectable arms 308A, 308B enable such selective connection to the hub of a catheter adapter, and a blunt cannula 310 extends from a distal end of the lever lock 305, with the blunt cannula 310 including a lumen extending therethrough so as to fluidly couple the fluid transfer device 300 and the catheter adapter (not shown) when interconnected.

Referring still to FIGS. 3A and 3B, the lever lock 305 further includes an elongated coupler portion 306, with coupler portion 306 being sized and configured to fit within a distal end portion of the introducer body 302. In some embodiments, the coupler portion 306 is substantially cylindrical and surrounds the lumen 311. However, it is to be understood that coupler portion 306 need not be cylindrical, and may be formed of any appropriate shape or shapes. In accordance with an aspect of the present disclosure, and as shown in FIGS. 3A and 3B, fluid transfer device 300 is configured such that the coupler portion 306 of the lever lock 305 is telescopically slidable within the distal portion of the introducer body so as to accommodate varying lengths of indwelling catheters. For example, referring to FIG. 3A, the lever lock 305 has a length E extending from the distal end 303 of introducer body 302 to the distal end of blunt cannula 310. However, referring to FIG. 3B, the lever lock 305 has a length F extending from the distal end 303 of introducer body 302 to the distal end of blunt cannula 310, with length F being greater than length E.

In some embodiments, the introducer body 302 may include an interior sidewall 304 which tapers inward as it approaches the distal end 303. With such a configuration, the lever lock 305 can be substantially prevented from being fully removed from the introducer body 302 when pulled, as the external surface of coupler portion 306 (and, particularly, a proximal end of the coupler portion 306) may form an interference fit with the interior sidewall 304 as the lever lock 305 is pulled to its maximum length. Additionally and/or alternatively, while not shown, the coupler portion 306 and/or the interior sidewall 304 may include detents and/or stop features to engage the lever lock 305 at desired positions relative to the introducer body 302. In some embodiments, the lever lock 305 may include a tab or other feature in order to engage a stop feature of the introducer body 302.

Accordingly, fluid transfer device 300 may be configured for usage with indwelling catheters having a variety of lengths. For example, referring to FIG. 3A, lever lock 305 may be positioned with an external overall length E so as to be utilized with longer indwelling catheters (e.g., 1.75 inch catheters), as the shorter lever lock length allows for a greater length of flow tube 312 to be fed from the fluid transfer device 300 and through the catheter adapter such that a distal end 313 of the flow tube 312 extends suitably beyond the indwelling catheter. Conversely, referring to FIG. 3B, lever lock 305 may be pulled outward so as to provide a longer external overall length F, enabling the fluid transfer device 300 to be utilized with shorter indwelling catheters (e.g., 1.00 inch catheters). In this way, the dimensions of the introducer body 302 and/or flow tube 312 need not change in order to accommodate various lengths of indwelling catheters.

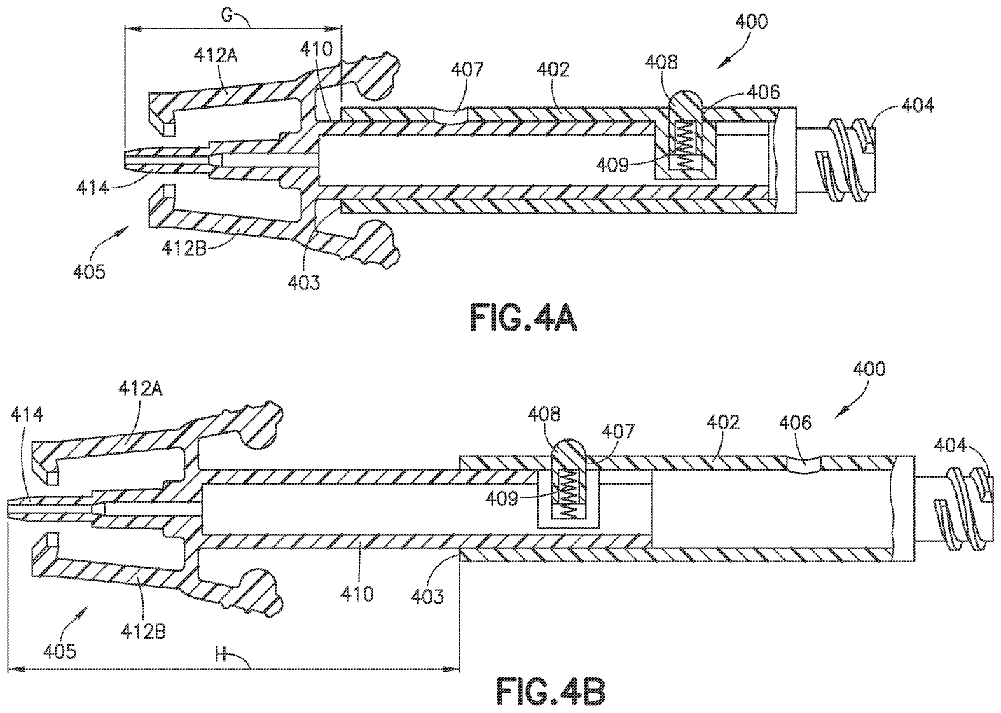

Next, referring to FIGS. 4A and 4B, a fluid transfer device 400 in accordance with another aspect of the present disclosure is illustrated. Fluid transfer device 400 includes an introducer body 402 having a distal end 403 and a proximal end 404. The proximal end 404 may be configured to include an interface (e.g., a threaded interface) for coupling the fluid transfer device 400 to, e.g., a luer lock access device (LLAD) for performing blood collection. However, it is to be understood that the proximal end of introducer body 402 may have any appropriate interface and/or coupling means.

Furthermore, it is to be understood that fluid transfer device 400 may be configured to include an actuator member movable with respect to the introducer body 402. In some embodiments, the actuator may be configured to be slidable along at least part of the length of the introducer body 402, with the actuator also being coupled to a flexible flow tube (not shown) such that movement of the actuator along the introducer body 402 results in corresponding linear movement of the flow tube relative to the introducer body 402. However, the actuator is not limited to such a slidable configuration, and any suitable actuator mechanism may be utilized.

The fluid transfer device 400 further includes a lever lock 405. Lever lock 405 is configured to removably couple the fluid transfer device 400 to, e.g., the hub of a catheter adapter. Deflectable arms 412A, 412B enable such selective connection to the hub of a catheter adapter, and a blunt cannula 414 extends from a distal end of the lever lock 405, with the blunt cannula 414 including a lumen extending therethrough so as to fluidly couple the fluid transfer device 400 and the catheter adapter (not shown) when interconnected.

The lever lock 405 further includes an elongated coupler body 410, with coupler body 410 being sized and configured slidably move within an interior of introducer body 402. In some embodiments, the coupler body 410 is substantially cylindrical. However, it is to be understood that neither the coupler body 410 nor the introducer body need to be cylindrical, and may be formed of any appropriate shape or shapes.

Referring still to FIGS. 4A and 4B, the lever lock 405 may further include a pin 408 biased by a spring 409 and extending transversely from a proximal portion of the coupler body 410. Furthermore, the introducer body 402 may include at least two openings 406, 407 formed therein, with the first opening 406 positioned near a proximal end portion of the introducer body 402, and the second opening 407 positioned nearer a distal end portion of the introducer body 402. Both the first opening 406 and second opening 407 are aligned and sized so as to capture the spring-biased pin 408 when the lever lock 405 is moved to a position proximate to either of the respective openings 406, 407. In this way, the engagement between the pin 408 and the openings 406, 407 provides a stop for the lever lock 405, limiting further axial movement of the lever lock 405 unless and until a user manually depresses the spring-biased pin 408 and pushes or pulls the lever lock 405 relative to the introducer body 402.

In accordance with an aspect of the present disclosure, and as shown in FIGS. 4A and 4B, fluid transfer device 400 is configured such that the coupler body 410 of the lever lock 405 is telescopically slidable within the introducer body 402 so as to accommodate varying lengths of indwelling catheters, with the lever lock 405 being selectively secured in a desired position by the pin 408 and respective openings 406, 407. For example, referring to FIG. 4A, the lever lock 405 has a length G extending from the distal end 403 of introducer body 402 to the distal end of blunt cannula 414. This position is “locked” by engagement of the spring-biased pin 408 with the first opening 406. Alternatively, referring to FIG. 4B, the lever lock 405 has a length H extending from the distal end 403 of introducer body 402 to the distal end of blunt cannula 414, with length H being greater than length G. This particular position is “locked” by engagement of the spring-biased pin 408 with the second opening 407. Accordingly, fluid transfer device 400 may be configured for usage with indwelling catheters having a variety of lengths. For example, referring to FIG. 4A, lever lock 405 (and pin 408) may be positioned relative to first opening 406 to provide external overall length G so as to be utilized with longer indwelling catheters (e.g., 1.75 inch catheters), as the shorter lever lock length allows for a greater length of flow tube to be fed from the fluid transfer device 400, through the catheter adapter, and beyond the indwelling catheter. Conversely, referring to FIG. 4B, lever lock 405 (and pin 408) may be positioned relative to second opening 407 so as to provide a longer external overall length H, enabling the fluid transfer device 400 to be utilized with shorter indwelling catheters (e.g., 1.00 inch catheters).

While the embodiment shown in FIGS. 4A and 4B only illustrate introducer body 402 as having two openings 406, 407, it is to be understood that one or more additional openings may be provided on introducer body 402, thereby accommodating other lengths of indwelling catheters (e.g., 1.25 inch catheters).

Additionally, while not shown in FIGS. 4A and 4B, it is to be understood that labels or other indicia may be provided on the introducer body 402 proximate the respective openings 406, 407 in order to indicate the length of indwelling catheter and/or catheter type corresponds to which opening. Furthermore, in some embodiments, the introducer body 402 may incorporate a “default” or “baseline” opening at a proximal end portion thereof, with the baseline opening not corresponding to any length of indwelling catheter and/or catheter type. In this way, the lever lock 405 can be set to the baseline position prior to any use, thereby requiring the user to specifically adjust the lever lock 405 to the desired position, which helps to ensure that the correct position of lever lock 405 is chosen relative to the actual indwelling catheter length and/or catheter type.

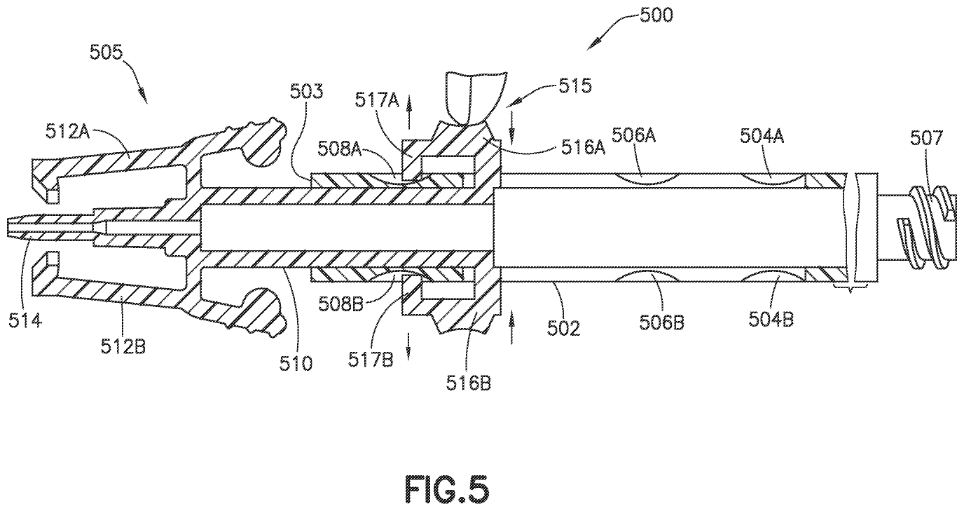

Referring now to FIG. 5, a fluid transfer device 500 in accordance with another aspect of the present disclosure is illustrated. Fluid transfer device 500 includes an introducer body 502 having a distal end 503 and a proximal end 507. While not shown in FIG. 5, it is to be understood that the proximal end 507 may be configured to include an interface (e.g., a threaded interface) for coupling the fluid transfer device 500 to, e.g., a luer lock access device (LLAD) for performing blood collection. However, it is to be understood that the proximal end 507 of introducer body 502 may have any appropriate interface and/or coupling means.

Additionally, while not shown, it is to be understood that fluid transfer device 500 may be configured to include an actuator member movable with respect to the introducer body 502. In some embodiments, the actuator may be configured to be slidable along at least part of the length of the introducer body 502, with the actuator also being coupled to a flexible flow tube (not shown) such that movement of the actuator along the introducer body 502 results in corresponding linear movement of the flow tube relative to the introducer body 502. However, the actuator is not limited to such a slidable configuration, and any suitable actuator mechanism may be utilized.

The fluid transfer device 500 further includes a lever lock 505. Lever lock 505 is configured to removably couple the fluid transfer device 500 to, e.g., the hub of a catheter adapter. Deflectable arms 512A, 512B enable such selective connection to the hub of a catheter adapter, and a blunt cannula 514 extends from a distal end of the lever lock 505, with the blunt cannula 514 including a lumen extending therethrough so as to fluidly couple the fluid transfer device 500 and the catheter adapter (not shown) when interconnected.

The lever lock 505 further includes an elongated coupler body 510, with coupler body 510 being sized and configured slidably move within an interior of introducer body 502. In some embodiments, the coupler body 510 is substantially cylindrical. However, it is to be understood that neither the coupler body 510 nor the introducer body need to be cylindrical, and may be formed of any appropriate shape or shapes.

Referring still to FIGS. 5, the lever lock 505 may be coupled to a cantilever slider member 515, with the cantilever slider member 515 extending transversely from a proximal portion of the coupler body 510. While not shown, the introducer body 502 may include a pair of slots formed along an axial length thereof, with the slots configured to accommodate respective coupling arms 516A, 516B of the cantilever slider member 515. This configuration of the cantilever slider member 515 allows the cantilever slider member 515 to be pinched or otherwise manually manipulated such that hook-like engagement portions 517A, 517B are capable of being engaged or disengaged, via cantilever action, from respective openings formed in the introducer body 502, as will be described in further detail below.

As shown in FIG. 5, the introducer body 502 may include opposing pairs of openings 504A, 504B, 506A, 506B, 508A, 508B formed therein. The first pair of opposing openings 504A, 504B may be positioned near a proximal end portion of the introducer body 502, the third pair of opposing openings 508A, 508B may be positioned nearer a distal end portion of the introducer body 502, and the second pair of opposing openings 506A, 506B may be positioned therebetween. The opposing pairs of openings 504A, 504B, 506A, 506B, 508A, 508B are aligned and sized so as to capture the respective engagement portions 517A, 517B of cantilever slider member 515 when the lever lock 505 is moved to a position proximate to openings 504A, 504B, 506A, 506B, 508A, 508B. In this way, the engagement between the engagement portions 517A, 517B and the openings 504A, 504B, 506A, 506B, 508A, 508B provides a stop for the lever lock 505, limiting further axial movement of the lever lock 505 unless and until a user manually actuates the cantilever slider member 515 and pushes or pulls the lever lock 505 relative to the introducer body 402.

As is shown in FIG. 5, fluid transfer device 500 is configured such that the coupler body 510 of the lever lock 505 is telescopically slidable within the introducer body 502 so as to accommodate varying lengths of indwelling catheters, with the lever lock 505 being selectively secured in a desired position by engagement portions 517A, 517B and the openings 504A, 504B, 506A, 506B, 508A, 508B. For example, in the position shown in FIG. 5, the lever lock 505 extends from a distal end 503 at a position suitable for usage with indwelling catheters a relatively short length (e.g., 1.00 inch catheters), as the longer lever lock length allows for a reduced length of flow tube to be fed from the fluid transfer device 500, through the catheter adapter, and beyond the indwelling catheter. Conversely, if lever lock 505 (and cantilever slider member 515) are positioned relative to openings 504A, 504B and/or 506A, 506B so as to provide a shorter external overall length of the lever lock 505, the fluid transfer device 500 may be utilized with longer indwelling catheters (e.g., 1.25 inch catheters, 1.75 inch catheters, etc.).

While the embodiment shown in FIG. 5 only illustrates introducer body 502 as having three opening pairs 504A, 504B, 506A, 506B, 508A, 508B, it is to be understood that more or fewer opening pairs may be provided on introducer body 502. Additionally, while not shown in FIG. 5, it is to be understood that labels or other indicia may be provided on the introducer body 502 proximate the respective opening pairs 504A, 504B, 506A, 506B, 508A, 508B in order to indicate the length of indwelling catheter and/or catheter type corresponds to which opening pair. Furthermore, in some embodiments, the introducer body 502 may incorporate a “default” or “baseline” opening pair at a proximal end portion thereof, with the baseline opening pair not corresponding to any length of indwelling catheter and/or catheter type. In this way, the lever lock 505 can be set to the baseline position prior to any use, thereby requiring the user to specifically adjust the lever lock 505 to the desired position, which helps to ensure that the correct position of lever lock 505 is chosen relative to the actual indwelling catheter length and/or catheter type.

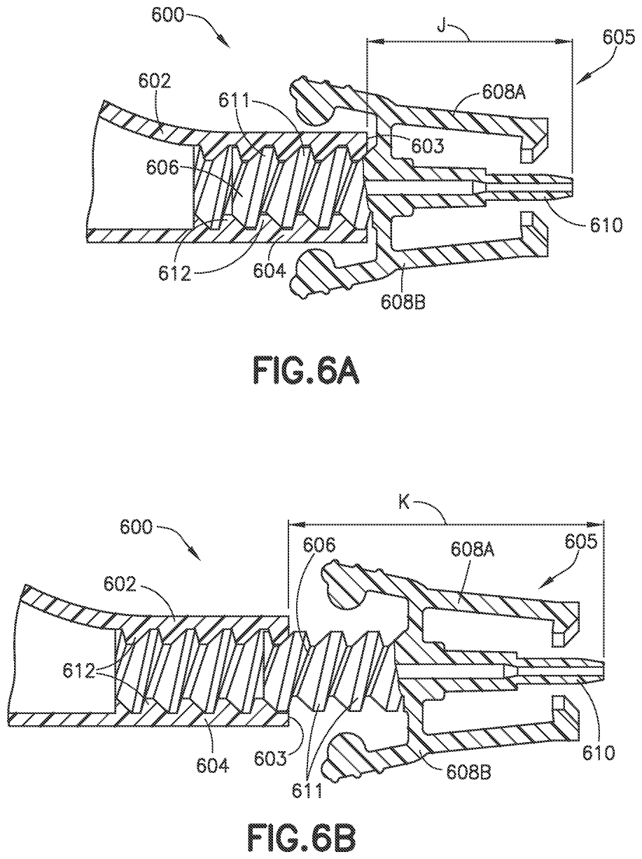

Referring now to FIGS. 6A and 6B, a fluid transfer device 600 in accordance with another aspect of the present disclosure is shown. Fluid transfer device 600 includes an introducer body 602 having a distal end 603 and a proximal end (not shown). The proximal end may be configured to include an interface (e.g., a threaded interface) for coupling the fluid transfer device 600 to, e.g., a luer lock access device (LLAD) for performing blood collection. However, it is to be understood that the proximal end of introducer body 602 may have any appropriate interface and/or coupling means.

Furthermore, while not shown, it is to be understood that fluid transfer device 600 may be configured to include an actuator member movable with respect to the introducer body 602. In some embodiments, the actuator may be configured to be slidable along at least part of the length of the introducer body 602, with the actuator also being coupled to a flexible flow tube (not shown) such that movement of the actuator along the introducer body 602 results in corresponding linear movement of the flow tube relative to the introducer body 602. However, the actuator is not limited to such a slidable configuration, and any suitable actuator mechanism may be utilized.

The fluid transfer device 600 further includes a lever lock 605. Lever lock 405 is configured to removably couple the fluid transfer device 600 to, e.g., the hub of a catheter adapter. Deflectable arms 608A, 608B enable such selective connection to the hub of a catheter adapter, and a blunt cannula 610 extends from a distal end of the lever lock 605, with the blunt cannula 610 including a lumen extending therethrough so as to fluidly couple the fluid transfer device 600 and the catheter adapter (not shown) when interconnected.

The lever lock 605 further includes an elongated coupler body 606. As shown in FIGS. 6A and 6B, the coupler body 606 includes a plurality of external threads 611 extending along at least a portion of its length. Additionally, the introducer body 602 includes a proximal portion 604 having a plurality of internal threads 612, with the internal threads 612 of proximal portion 604 being sized and configured to receive the external threads 611 of coupler body 606. In some embodiments, both the coupler body 606 and the proximal portion 604 are substantially cylindrical.

In accordance with an aspect of the present disclosure, and as shown in FIGS. 6A and 6B, fluid transfer device 600 is configured such that the coupler body 606 of the lever lock 605 is axially translatable relative to the introducer body 602 so as to accommodate varying lengths of indwelling catheters, with the lever lock 605 being selectively extended to a desired position by way of clockwise or counterclockwise rotation of the lever lock 605 relative to the introducer body 602. For example, referring to FIG. 6A, the lever lock 605 has a length J extending from the distal end 603 of introducer body 602 to the distal end of blunt cannula 610. Alternatively, referring to FIG. 6B, the lever lock 605 has a length K extending from the distal end 603 of introducer body 602 to the distal end of blunt cannula 610, with length K being greater than length J.

Accordingly, fluid transfer device 600 may be configured for usage with indwelling catheters having a variety of lengths. For example, referring to FIG. 4A, lever lock 605 may be positioned relative to the distal end 603 of introducer body 602 to provide external overall length J so as to be utilized with longer indwelling catheters (e.g., 1.75 inch catheters), as the shorter lever lock length allows for a greater length of flow tube (not shown) to be fed from the fluid transfer device 600, through the catheter adapter, and beyond the indwelling catheter. Conversely, referring to FIG. 6B, lever lock 605 may be positioned relative to the distal end 603 of introducer body 602 to provide external overall length K, enabling the fluid transfer device 600 to be utilized with shorter indwelling catheters (e.g., 1.00 inch catheters).

While not shown in FIGS. 6A and 6B, it is to be understood that the introducer body 602 and/or the lever lock 605 may include one or more detents and/or stops configured to limit the adjustment positions of the lever lock 605 relative to the introducer body 602. Additionally and/or alternatively, the coupler body 606 of lever lock 605 may include markings and/or other indicia so as to provide guidance to the user as to proper positioning of the lever lock 605 for use with a particular length of indwelling catheter.

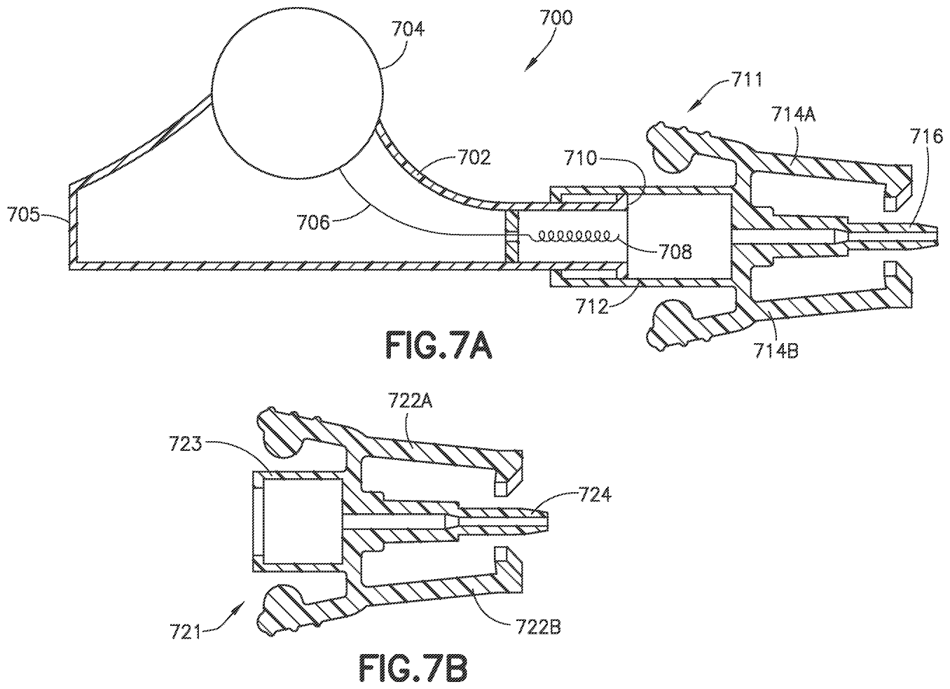

Next, referring to FIGS. 7A and 7B, a fluid transfer device 700 in accordance with another aspect of the present disclosure is illustrated. Fluid transfer device 700 includes an introducer body 702 having a distal end 710 and a proximal end 705. While not shown, the proximal end 705 may be configured to include an interface (e.g., a threaded interface) for coupling the fluid transfer device 700 to, e.g., a luer lock access device (LLAD) for performing blood collection. However, it is to be understood that the proximal end 705 may have any appropriate interface and/or coupling means.

As shown in FIG. 7A, fluid transfer device 700 may include an actuator portion 704 movable with respect to the introducer body 702. Specifically, actuator portion 704 may include a rotary actuator that is rotatable in both a clockwise and counterclockwise direction relative to the introducer body. The actuator portion 704 is coupled to a flexible flow tube 706 such that rotational movement of the actuator portion 704 results in corresponding linear movement of the flow tube 706 relative to the introducer body 702. In this way, the flow tube 706 may be fed through and/or retracted into the fluid transfer device 700 such that a distal end 708 of flow tube 706 may extend beyond an indwelling catheter. However, it is to be understood that other forms of actuators and/or methods of actuation of the flow tube 706 relative to the introducer body 702 may also be utilized in accordance with the present disclosure.

Referring still to FIG. 7A, the fluid transfer device 700 also includes a first lever lock 711. First lever lock 711 is configured to removably couple the fluid transfer device 700 to, e.g., the hub of a catheter adapter. Deflectable arms 714A, 714B enable such selective connection to the hub of a catheter adapter, and a blunt cannula 716 extends from a distal end of the first lever lock 711, with the blunt cannula 716 including a lumen (not shown) extending therethrough so as to fluidly couple the fluid transfer device 700 and the catheter adapter (not shown) when interconnected.

The first lever lock 711 further includes an elongated coupler portion 712, with the coupler portion 712 being configured to be removably coupled to a distal end portion of the introducer body 702. In some embodiments, the coupler portion 712 is substantially cylindrical. However, it is to be understood that coupler portion 712 need not be cylindrical, and may be formed of any appropriate shape or shapes. The coupler portion 712 may be selectively coupled to the introducer body 702 via any appropriate attachment method or mechanism such as, e.g., an interference fit, a cantilever snap fit, an annular snap fit, one or more magnets, a threaded interface, etc.

As noted above, due to variations in the length of indwelling catheters, in some instances, a flow tube may not sufficiently extend beyond the distal end of the indwelling catheter or, alternatively, may extend an undesirable distance beyond the distal end of the indwelling catheter. Thus, in accordance with an aspect of the present disclosure, and as shown in FIGS. 7A and 7B, fluid transfer device 700 may be provided and/or packaged with a plurality of different lever locks, with each lever lock configured to accommodate a specific length of indwelling catheter. For example, referring to FIG. 7A, the first lever lock 711 has a coupler portion 712 configured to extend a first length beyond the distal end 710 of introducer body 702. However, referring to FIG. 7B, an entirely separate, second lever lock 721 may be provided. Like first lever lock 711, second lever lock 721 includes deflectable arms 722A, 722B to enable selective connection of the second lever lock 721 to the hub of a catheter adapter, and a blunt cannula 724 extends from a distal end of the second lever lock 721 to fluidly couple the fluid transfer device 700 and the catheter adapter (not shown) when interconnected. The second lever lock 721 may also include a coupler portion 723, with coupler portion 723 capable of selective coupling to the introducer body 702 via any appropriate attachment method or mechanism such as, e.g., an interference fit, a cantilever snap fit, an annular snap fit, one or more magnets, a threaded interface, etc.

As is shown in FIGS. 7A and 7B, coupler portion 722 is shorter than the coupler portion 712 of first lever lock 721. In this way, fluid transfer device 700 may be provided with a variety of lever locks for usage with indwelling catheters having a variety of lengths. For example, referring to FIG. 7A, first lever lock 711 may be utilized with shorter indwelling catheters (e.g., 1.00 inch catheters), as the longer coupler portion 712 of first lever lock 711 allows for a shorter length of flow tube 706 to be fed from the fluid transfer device 700, through the catheter adapter, and through (and beyond) the indwelling catheter. Conversely, referring to FIG. 7B, second lever lock 721 may be utilized with longer indwelling catheters (e.g., 1.75 inch catheters), as the shorter coupler portion 723 allows a longer length of flow tube 706 to be fed from the fluid transfer device 700. In this way, the dimensions of the introducer body 702 and/or flow tube 706 need not change in order to accommodate various lengths of indwelling catheters.

In some embodiments, the fluid transfer device 700 is packaged with multiple lever locks of various sizes and configurations, thereby enabling the user to select the appropriate lever lock for connection to the introducer body dependent upon, e.g., indwelling catheter length, application, etc. While FIGS. 7A and 7B only illustrate lever locks having two differing lengths, it is to be understood that the fluid transfer device 700 may be provided with more than two lever locks of different so as to accommodate other indwelling catheter lengths such as, e.g., 1.25 inches.

Additionally and/or alternatively, the first lever lock 711 and/or second lever lock 721 may be provided with markings and/or other indicia so as to provide guidance to the user as to proper selection of the lever lock(s) for use with a particular length of indwelling catheter and/or application. For example, first lever lock 711 may be labeled “1.00”, signifying its intended use with indwelling catheters having a length of 1.00 inches, while second lever lock 721 may be labeled “1.75”, signifying its intended use with indwelling catheters having a length of 1.75 inches.

While several embodiments of fluid transfer devices configured for blood draw during catheter indwell were described in the foregoing detailed description, those skilled in the art may make modifications and alterations to these embodiments without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are embraced within their scope.

What is claimed is:

1. A fluid transfer device for use with a peripheral intravenous catheter (PIVC) comprising:
an introducer body having a proximal end and a distal end;
a flow tube at least partially housed within the introducer body, wherein the flow tube is configured to be selectively extendable from the introducer body; and
a lever lock coupled to the distal end of the introducer body, wherein the lever lock includes a lumen configured to allow the flow tube to pass therethrough, and wherein an effective length of the lever lock relative to the distal end of the introducer body is adjustable in order to accommodate use of the fluid transfer device with indwelling catheters of the PIVC having various lengths.

2. The fluid transfer device of claim 1, wherein the lever lock comprises an elongated coupler portion, a pair of deflectable arms, and a blunt cannula.

3. The fluid transfer device of claim 2, wherein the pair of deflectable arms are configured to releasably couple the lever lock to a hub of a catheter adapter of the PIVC.

4. The fluid transfer device of claim 2, wherein at least a portion of the elongated coupler portion is configured to be slidable within at least a distal portion of the introducer body.

5. The fluid transfer device of claim 4, wherein the distal portion of the introducer body tapers so as to restrict an amount in which the elongated coupler portion of the lever lock may slide relative to the distal portion of the introducer.

6. The fluid transfer device of claim 2, wherein the elongated coupler portion of the lever lock comprises a plurality of external threads, and wherein a distal portion of the introducer body comprises a plurality of internal threads engageable with the external threads of the elongated coupler portion.

7. The fluid transfer device of claim 2, further comprising a spring-biased pin coupled to the elongated coupler portion of the lever lock.

8. The fluid transfer device of claim 7, wherein the introducer body further comprises at least two openings formed therein, and wherein the spring-biased pin of the elongated coupler portion of the lever lock is configured to selectively engage with the at least two openings of the introducer body.

9. The fluid transfer device of claim 2, further comprising a cantilever slider member coupled to a proximal portion of the elongated coupler portion of the lever lock, wherein the cantilever slider member comprises a pair of engagement portions.

10. The fluid transfer device of claim 9, wherein the introducer body further comprises at least two opening pairs formed therein, and wherein the pair of engagement portions of the cantilever slider member are configured to selectively engage with the at least two opening pairs of the introducer body.

11. The fluid transfer device of claim 1, further comprising an actuator member movable relative to the introducer body, wherein the actuator member is configured to feed the flow tube through the introducer body and the lever lock.

12. The fluid transfer device of claim 1, wherein the proximal end of the introducer body comprises a threaded interface.

13. A fluid transfer device for use with a peripheral intravenous catheter (PWC)comprising:
an introducer body having a proximal end and a distal end;
a flow tube at least partially housed within the introducer body, wherein the flow tube is configured to be selectively extendable from the introducer body;
a first lever lock selectively couplable to the distal end of the introducer body, wherein the first lever lock includes a first lumen configured to allow the flow tube to pass therethrough; and
a second lever lock selectively couplable to the distal end of the introducer body, wherein the second lever lock includes a second lumen configured to allow the flow tube to pass therethrough,
wherein a length of the first lever lock is shorter than a length of the second lever lock in order to accommodate use of the fluid transfer device with indwelling catheters of the PIVC having various lengths.

14. The fluid transfer device of claim 13, wherein the each of the first lever lock and the second lever lock comprises an elongated coupler portion, a pair of deflectable arms, and a blunt cannula.

15. The fluid transfer device of claim 14, wherein the pair of deflectable arms are configured to releasably couple the lever lock to a hub of a catheter adapter of the PIVC.

16. The fluid transfer device of claim 13, wherein the first lever lock is configured for use with indwelling catheters having a length of 1.00 inches, and the second lever lock is configured for use with indwelling catheters having a length of 1.75 inches.

17. A fluid transfer device for use with a peripheral intravenous catheter (PIVC)comprising:
an introducer body having a proximal end and a distal end;
a flow tube at least partially housed within the introducer body, wherein the flow tube is configured to be selectively extendable from the introducer body; and
a lever lock coupled to the distal end of the introducer body, wherein the lever lock comprises an elongated coupler portion, and further wherein the elongated coupler portion of the lever lock is telescopically slidable within at least a distal portion of the introducer body in order to accommodate use of the fluid transfer device with indwelling catheters of the PIVC having various lengths.

18. The fluid transfer device of claim 17, wherein the lever lock further comprises a pair of deflectable arms and a blunt cannula, and wherein the pair of deflectable arms are configured to releasably couple the lever lock to a hub of a catheter adapter of the PIVC.

19. The fluid transfer device of claim 17, further comprising a spring-biased pin coupled to the elongated coupler portion of the lever lock, wherein the introducer body further comprises at least two openings formed therein, and wherein the spring-biased pin of the elongated coupler portion of the lever lock is configured to selectively engage with the at least two openings of the introducer body.

20. The fluid transfer device of claim 17, further comprising a cantilever slider member coupled to a proximal portion of the elongated coupler portion of the lever lock, wherein the cantilever slider member comprises a pair of engagement portions, wherein the introducer body further comprises at least two opening pairs formed therein, and wherein the pair of engagement portions of the cantilever slider member are configured to selectively engage with the at least two opening pairs of the introducer body.
